(19) United States
(12) Patent Application Publication
(10) Pub. No.: US 2016/0287897 A1
(43) Pub. Date: Oct. 6, 2016

(54) SYSTEMS AND METHODS FOR IRRADIANCE CONTROL OF A SET OF LIGHT-EMITTING DIODES

(71) Applicant: KONINKLIJKE PHILIPS N.V., Eindhoven (NL)

(72) Inventor: Siegfried Walter KAESTLE, NUFFRINGEN (NL)

(21) Appl. No.: 15/037,357

(22) PCT Filed: Nov. 14, 2014
(86) PCT No.: PCT/IB2014/066036

§ 371 Date: May 18, 2016

Related U.S. Application Data
(60) Provisional application No. 61/908,191, filed on Nov. 25, 2013.

(51) Int. Cl. A61N 5/06; H05B 33/08; H05B 37/02

(52) U.S. Cl. CPC A61N 5/0621; H05B 37/0227; H05B 33/0851; A61N 2005/0626; A61N 2005/0638; A61N 2005/0652

(57) ABSTRACT

Systems and methods for adjusting the amount of power supplied to light sources in a phototherapy panel are configured to maintain irradiance at a level that corresponds to a recommended level. By virtue of such adjustments, infants with jaundice can be treated and/or a notification that the light sources need to be replaced maybe determined.

### BACKGROUND

1. Field

The present disclosure pertains to systems and methods for controlling current being supplied to light sources, and, in particular, to systems and methods that control and/or provide a panel of light emitting diodes that is suitable to treat jaundice in infants by maintaining irradiance at a level corresponding to a recommended level.

2. Description of the Related Art

Infants, e.g. neonates, may be treated with phototherapy. An example of phototherapy is jaundice treatment using a panel of light sources that emit, e.g., blue light.

### SUMMARY

Accordingly, it is an object of one or more embodiments of the present invention to provide a system. The system includes a set of light sources, a photo-sensor, and one or more processors. The set of light sources is configured to emit electromagnetic radiation. The set includes light-emitting diodes. The photo-sensor is configured to generate output signals conveying information related to an irradiance level of electromagnetic radiation emitted by a subset of light sources from the set of light sources. The one or more processors are configured to obtain a recommended therapy regimen of electromagnetic radiation for a subject, to determine an irradiance parameter of electromagnetic radiation emitted by the subset of light sources, wherein the irradiance parameter is based on the generated output signals, and to adjust an amount of power supplied to the set of light sources. The adjustments are based on the recommended therapy regimen and the determined irradiance parameter

It is yet another aspect of one or more embodiments of the present invention to provide a method of adjusting an amount of power supplied to a set of light sources. The method includes activating a set of light sources, wherein the set includes light-emitting diodes (LEDs); emitting, by the set of light sources, electromagnetic radiation responsive to activation; generating output signals conveying information related to an irradiance level of electromagnetic radiation emitted by a subset of light sources from the set of light sources; obtaining a recommended therapy regimen of electromagnetic radiation for a subject; determining an irradiance parameter of electromagnetic radiation emitted by the subset of light sources, wherein the irradiance parameter is based on the output signals; and adjusting an amount of power supplied to the set of light sources, wherein adjustments are based on the recommended therapy regimen and the determined irradiance parameter.

It is yet another aspect of one or more embodiments to provide a system configured to adjust an amount of power supplied to a set of light source. The system includes emitting means for emitting electromagnetic radiation, wherein the emitting means includes light-emitting diodes (LEDs); means for generating output signals conveying information related to an irradiance level of electromagnetic radiation emitted by a portion of the emitting means; means for obtaining a recommended therapy regimen of electromagnetic radiation for a subject; means for determining an irradiance parameter of electromagnetic radiation emitted by the portion of the emitting means, wherein the irradiance parameter is based on the output signals; and means for adjusting an amount of power supplied to the means for emitting, wherein adjustments are based on the recommended therapy regimen and the determined irradiance parameter.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 illustrates a schematic view of a phototherapy panel in accordance with one or more embodiments;

FIG. 2 illustrates a top view of a phototherapy panel in accordance with one or more embodiments;

FIG. 3 schematically illustrates a system in accordance with one or more embodiments;

FIG. 4 illustrates a circuit diagram for a phototherapy panel in accordance with one or more embodiments; and

FIG. 5 illustrates a method for adjusting the amount of power supplied to a set of light sources in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

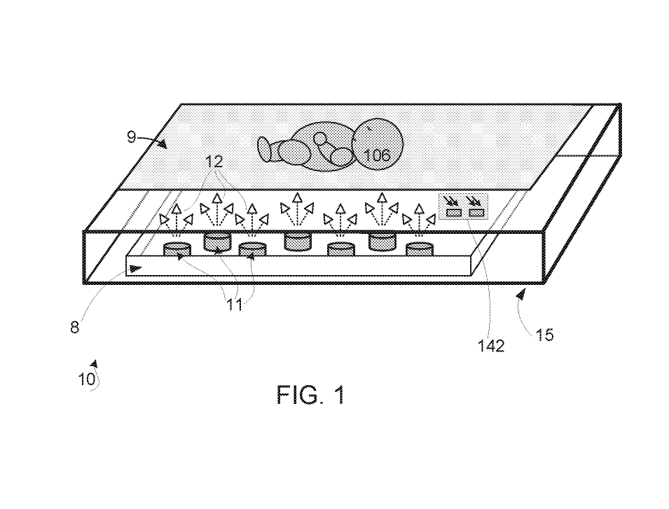

FIG. 1 illustrates a system 10 that includes one or more of a phototherapy panel 15, a set of light sources 11, one or more (photo-)sensors 142, and/or other components (some of which may be depicted in other figures). Phototherapy panel 15 (interchangeably referred to as panel 15) may be configured to provide phototherapy to an infant 106. System 10 can be integrated, embedded, incorporated, combined, and/or otherwise operating in conjunction with an incubator, baby warmer, and/or medical apparatus used for treatment of infants. Panel 15 and/or system 10 may include an infant-supporting body 9, and/or other components. In some embodiments, infant-supporting body 9 may include the top surface of panel 15. System 10 may include a body 8 configured to hold and/or carry set of light sources 11. In some embodiments, body 8 includes a (removable) tray, rack, and/or other structure suitable to hold and/or carry set of light sources 11. As used herein, a single light source may be referred to as light source 11, a set of light sources may be referred to as set of light sources 11, and either may be referred to as light source(s) 11, to be interpreted by the context of the particular usage in this disclosure. Although the number of light sources 11 depicted in FIG. 1 is definite, this depiction is merely exemplary, as are all figures, and any number of light sources may be included in system 10. In some embodiments, set of light sources 11 includes more than three light sources. As used herein, photo-sensor 142 may be interchangeably referred to as sensor 142.

Phototherapy can be used to treat jaundice (or hyperbilirubinemia) by reducing the level of bilirubin. Effective and/or appropriate levels of phototherapy may be based on an infant's age, size, weight, and/or other physiological, environmental, and/or infant-specific parameters. Phototherapy uses electromagnetic radiation having a peak wavelength between, e.g., 460 nm and 500 nm, an emission spectrum ranging from, e.g., 400 nm to 520 nm, and preferably using a narrow bandwidth delivered at an irradiance of, e.g., 15-35·W/cm²/nm to, e.g., up to 80% of an infant's body surface area (BSA). Phototherapy may potentially need to be kept from directly impinging on the eyes of the infant, e.g. by making the infant wear goggles. In some embodiments, phototherapy may use electromagnetic radiation having a broad range of wavelengths/frequencies/colors, including radiation that does not provide effective treatment.

With usage, a light source 11 may have a reduced efficiency (e.g. compared to a similar but brand-new light source). Efficiency may e.g. be expressed as a ratio of supplied/used power and generated level of irradiance. As a result of reduced efficiency (e.g. developed gradually over time with hundreds or thousands of hours of usage), a light source may need more power and/or current to provide the same level of irradiance. In some cases, the efficiency of a particular light source may have been reduced to such an extent that the maximum amount of power and/or current that can (safely) be supplied would not be sufficient to maintain the same level of irradiance as a similar but brand-new light source. By virtue of the techniques described in this disclosure, a recommended level of irradiance for a set of light sources 11 may be automatically maintained by increasing the supplied power and/or current to counteract the reduced efficiency (or estimated reduced efficiency) of the set of light sources 11. System 10 may automatically indicate when one or more light sources need to be replaced based on measurements of the level of irradiance provided and/or generated by at least a subset of light sources 11.

Infant-supporting body 9 in FIG. 1 may include a transparent or translucent light emitting surface and may be configured such that infant 106 is positioned above and/or supported on a transparent or translucent light emitting surface during system operation. The transparent or translucent light emitting surface may engage infant 106 during use of system 10. In other words, infant 106 is placed on the transparent or translucent light emitting surface during use of panel 15 and/or system 10.

Set of light sources 11 may emit electromagnetic radiation 12 upon activation. Emitted electromagnetic radiation may be guided through infant-supporting body 9. Electromagnetic radiation 12 may impinge on infant 106 and thus provide phototherapy for infant 106 during operation of system 10. Individual light sources 11 may include one or more of a light-emitting diode (LED), an organic light-emitting diode (OLED), and/or other source of electromagnetic radiation. The LEDs may be configured to emit electromagnetic radiation in a narrow range suitable for phototherapy. In some embodiments, all light sources in the set of light sources may be LEDs. Infant 106 may be monitored while on or near system 10 or a component thereof, e.g. while undergoing phototherapy.

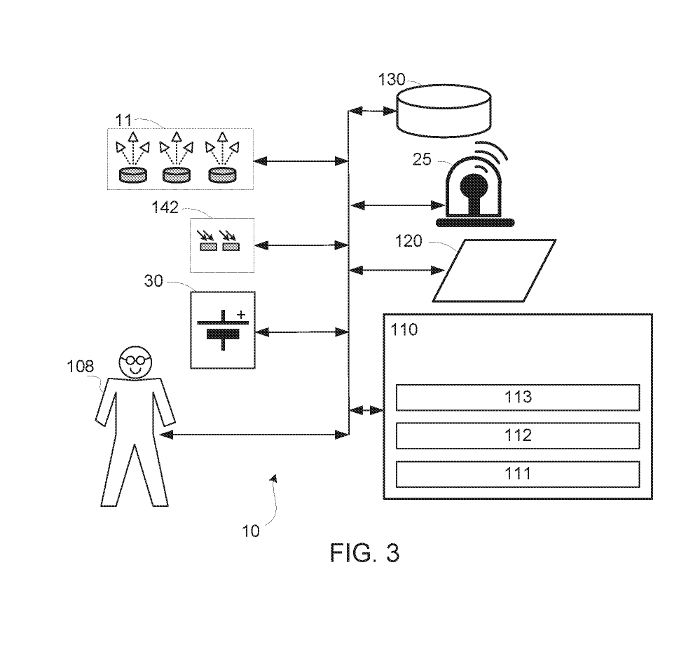

Set of light sources 11 may be arranged in a regular pattern, irregular pattern, or combination of both. For example, light sources 11 may be arranged in a regular grid. The grid includes N rows and M columns, wherein N and M may number between 2 and 40, and/or between other numbers. In a preferred embodiment, N and M are 6 and 21. In some embodiments, as schematically illustrated in FIG. 3, N and M may be 10 and 12. In some embodiments, light sources 11 are arranged in a regular and/or off-set grid to provide uniform electromagnetic radiation and/or phototherapy.

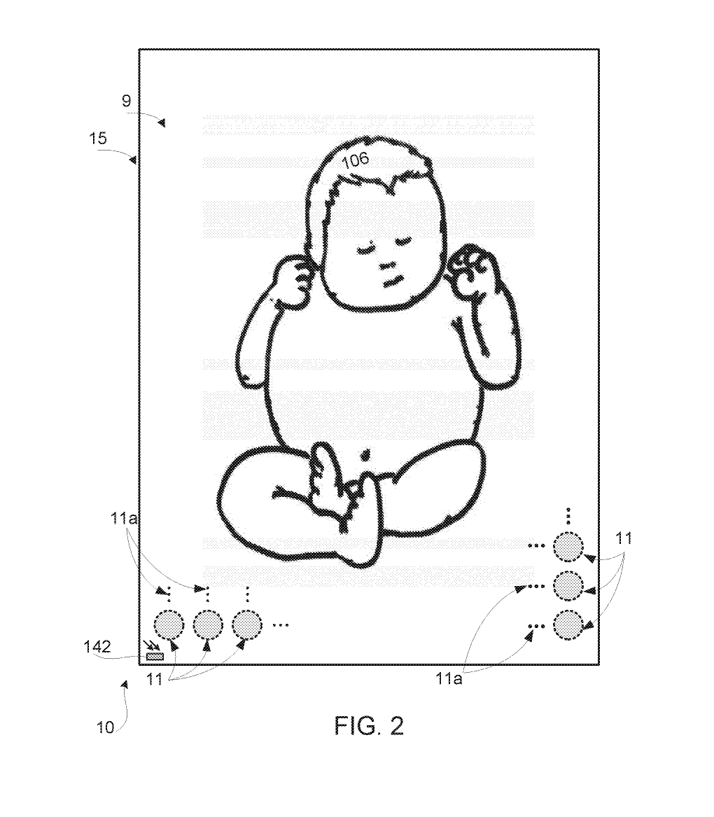

By way of illustration, FIG. 2 illustrates a top-view of panel 15. The depiction of three light sources 11 underneath the bottom left corner of infant-supporting body 9 is not meant to be limiting, but to be exemplary. The depiction of three light sources 11 underneath the bottom right corner of infant-supporting body 9 is not meant to be limiting, but to be exemplary. Ellipses 11a indicate additional light sources that may be arranged underneath infant-supporting body 9, horizontally, vertically, diagonally, and/or in multiple directions, e.g. to create a regular grid of light sources. Light sources 11 may extend underneath the entire infant-supporting body 9 of panel 15, including under infant 106.

Referring to FIG. 1, light sources 11 of system 10 may be configured to have one or more of a controllable level of intensity (e.g. denoted by a percentage of the maximum available level of intensity for an individual light source, interchangeably referred to as a level of irradiance), a controllable direction and/or angle of illumination (as depicted by multiple directions of electromagnetic radiation 12 for individual light sources in FIG. 1), a controllable selection of illumination spectra, and/or other controllable illumination characteristics and/or illumination parameters. For example, illumination parameters of one or more light sources 11 may be controlled by adjusting optical components within the light source, including, but not limited to, one or more of refractive components, reflective components, lenses, mirrors, filters, polarizers, diffraction gradients, optical fibers, and/or other optical components. Individual light sources 11 may be controlled such that only part of (the exposed skin of) infant 106 is illuminated. Alternatively, and/or simultaneously, illumination parameters of one or more light sources 11 may be controlled by adjusting the (amount of) power and/or current supplied to the light sources. For example, increasing the amount of current supplied to a particular light source 11 may increase the level of irradiance of the electromagnetic radiation emitted by this particular light source 11. Conversely, decreasing and/or reducing the amount of current supplied to a particular light source 11 may decrease and/or reduce the level of irradiance of the electromagnetic radiation emitted by this particular light source 11.

Sensor(s) 142 of system 10 in FIG. 1 and/or other figures are configured to generate output signals conveying information related to one or more parameters of system operation and/or electromagnetic radiation 12 emitted by one or more light sources 11. Parameters of system operation may include, by way of non-limiting example, the amount of power and/or current supplied to a light source 11, a set of light sources 11, or all light sources 11 within system 10. Parameters of electromagnetic radiation may include one or more of peak wavelength, wavelength band, emission spectrum, bandwidth, irradiance, luminance, intensity/irradiance and/or other parameters of electromagnetic radiation. Alternatively, and/or simultaneously, output signals (e.g. generated by various types of sensors) may convey information related to the age, position, posture, size, weight, bilirubin-level, and/or status of infant 106, physiological, environmental, and/or infant-specific (medical) parameters related to infant 106, and/or other information. System 10 may use any of the generated output signals as feedback to control operations of system 10, and/or to monitor infant 106. In some embodiments, the conveyed information is related to parameters associated with the state and/or condition of infant 106, the breathing of infant 106, the gas breathed by infant 106, the heart rate of infant 106, the respiratory rate of infant 106, vital signs of infant 106, including one or more temperatures, whether peripheral or central, and/or other parameters. Individual sensors or subsets of sensors from sensors 142 may be designated by specific functions, such as, e.g., an irradiance sensor, a light sensor, a temperature sensor, a flux sensor, and/or other sensors.

In some embodiments, sensors 142 are configured to generate output signals conveying information related to a level of bilirubin in infant 106. Such sensors can be used to perform interstitial fluids bilirubin measurements.

The illustration of sensor 142 including two members in FIG. 1 is not intended to be limiting. System 10 may include one or more sensors. Resulting signals or information from one or more sensors 142 are transmitted to other components of system 10. This transmission can be wired and/or wireless. Monitoring of infant 106 and/or the environment near infant 106 may be based on one or more sensors 142 and/or any of the related parameters described herein. Monitoring and/or measuring may be used as a contact-less, non-invasive means to obtain information. “Contact-less” refers to either refraining from the use of adhesives (e.g. on the skin of infant 106) and/or refraining from direct skin contact in the context of this disclosure. Note that any sensor described herein can be contact-less.

In some embodiments, the function of system 10 needs to be accomplished within an incubator environment, such that the micro-climate within the incubator (including one or more of an internal temperature, humidity, and/or other characteristics of a micro-climate within an incubator), is left substantially undisturbed during phototherapy. By way of illustration, FIG. 3 illustrates a system 10 for provide phototherapy for an infant, that may be embedded within an incubator. As depicted in FIG. 3, system 10 includes, in addition to any previously mentioned components in this disclosure, one or more of a set of light sources 11, one or more sensors 142, a power supply 30, an indicator 25, electronic storage 130, a user interface 120, one or more processors or controllers 110 (collectively referred to herein as processor 110), a parameter determination component 111, a control component 112, a therapy component 113, an alert component 114, and/or other components.

One or more power supplies 30 are configured to supply current and/or power to one or more light sources 11, and/or other components of system 10. As used herein, multiple power supplies in a system such as system 10 may be referred to as “power supply 30.” In preferred embodiments, power supply 30 may be configured to supply direct current rather than alternating current to the one or more light sources 11. By supplying direct current to the one or more light sources 11 upon activation of system 10, electromagnetic interference (EMI), particularly EMI having radio frequencies (RF) may be reduced compared to phototherapy systems that use alternating currents to power one or more light sources.

User interface 120 of system 10 in FIG. 3 is configured to provide an interface between system 10 and user 108 through which the user can provide information to and/or receive information from system 10. This enables data, results, and/or instructions and any other communicable items, collectively referred to as “information,” to be communicated between the user and system 10. An example of information that can be conveyed to user 108 is the current intensity/irradiance level of phototherapy, and/or the elapsed time since phototherapy commenced. Examples of interface devices suitable for inclusion in user interface 120 include a keypad, buttons, switches, a keyboard, knobs, levers, a display screen, a touch screen, speakers, a microphone, an indicator light, an audible alarm, and a printer. Information may be provided to user 108 by user interface 120 in the form of auditory signals, visual signals, tactile signals, and/or other sensory signals, or any combination thereof.

By way of non-limiting example, user interface 120 may include a radiation source capable of emitting light. The radiation source includes, for example, one or more of at least one LED, at least one light bulb, a display screen, and/or other sources. User interface 120 controls the radiation source to emit light in a manner that conveys information to user 108.

It is to be understood that other communication techniques, either hard-wired or wireless, are also contemplated herein as user interface 120. For example, in one embodiment, user interface 120 is integrated with a removable storage interface provided by electronic storage 130. In this example, information is loaded into system 10 from removable storage (e.g., a smart card, a flash drive, a removable disk, etc.) that enables the user(s) to customize the implementation of system 10. Other exemplary input devices and techniques adapted for use with system 10 as user interface 120 include, but are not limited to, an RS-232 port, RF link, an IR link, modem (telephone, cable, Ethernet, internet or other). In short, any technique for communicating information with system 10 is contemplated as user interface 120.

Electronic storage 130 of system 10 in FIG. 3 comprises electronic storage media that electronically stores information. The electronic storage media of electronic storage 130 may include one or both of system storage that is provided integrally (i.e., substantially non-removable) with system 10 and/or removable storage that is removably connectable to system 10 via, for example, a port (e.g., a USB port, a FireWire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EPROM, EEPROM, RAM, etc.), solid-state storage media (e.g., flash drive, etc.), and/or other electronically readable storage media. Electronic storage 130 may store software algorithms, information determined by processor 110, information received via user interface 120, and/or other information that enables system 10 to function properly. For example, electronic storage 130 may record or store information related to the provided phototherapy, and/or other information. Electronic storage 130 may be a separate component within system 10, or is provided integrally with one or more other components of system 10 (e.g., processor 110).

Processor 110 of system 10 in FIG. 3 is configured to provide information processing and control capabilities in system 10. As such, processor 110 includes one or more of a digital processor, a microcontroller, an analog processor, a digital circuit designed to process information, an analog circuit designed to process information, a state machine, and/or other mechanisms for electronically processing information. Although processor 110 is shown in FIG. 3 as a single entity, this is for illustrative purposes only. In some implementations, processor 110 includes a plurality of processing units.

As is shown in FIG. 3, processor 110 may be configured to perform one or more functions. For example, processor 110 may be configured to execute one or more computer program components. The one or more computer program components include one or more of parameter determination component 111, control component 112, therapy component 113, alert component 114, and/or other components. Processor 110 is configured to execute components 111, 112, 113, and/or 114 by software; hardware; firmware; some combination of software, hardware, and/or firmware; and/or other mechanisms for configuring processing capabilities on processor 110.

It should be appreciated that although components 111-114 are illustrated in FIG. 3 as being co-located within a single processing unit, in implementations in which processor 110 includes multiple processing units, one or more of components 111-114 may be located remotely from the other components. The description of the functionality provided by the different components 111-114 described below is for illustrative purposes, and is not intended to be limiting, as any of components 111-114 may provide more or less functionality than is described. For example, one or more of components 111-114 may be eliminated, and some or all of its functionality may be provided by other ones of components 111-114, and/or by processor 110. Note that processor 110 may be configured to execute one or more additional components that may perform some or all of the functionality attributed below to one of components 111-114.

Parameter determination component 111 of system 10 in FIG. 3 is configured to determine one or more irradiance parameters, status parameters, medical parameters, environmental parameters, and/or other parameters from output signals generated by one or more sensors 142. Environmental parameters are related to one or more of the parameters of electromagnetic radiation, various temperatures, humidity level, and/or other environmental parameters, which may be related to environmental conditions near system 10. One or more status parameters may be related to an infant's age, size, volume, weight, and/or other infant-specific parameters. One or more status parameters may be related to the presence, posture, and/or position of infant 106. One or more medical parameters may be related to monitored vital signs of infant 106, physiological parameters of infant 106, and/or other medical parameters of infant 106. Some or all of this functionality can be incorporated or integrated into other computer program components of processor 110.

Control component 112 of system 10 in FIG. 3 is configured to control and/or adjust one or more amounts of power supplied to one or more light sources 11 such that an amount of emitted electromagnetic radiation (in particular electromagnetic radiation 12) is adjustable through one or more settings. The emitted electromagnetic radiation is intended to provide phototherapy for infant 106. Adjustments and/or control by control component 112 may be organized based on individual light sources, one or more subsets of light sources, one or more groups of light sources, one or more rows and/or columns of light sources, and/or any combination thereof. Adjustments and/or control by control component 112 include adjustments and/or control of one or more of the controllable level of intensity/irradiance, the controllable direction and/or angle of illumination, the controllable selection of illumination spectra, and/or other controllable illumination characteristics and/or illumination parameters of one or more light sources 11. Adjustments and/or control by control component 112 may be based on any parameters determined by parameter determination component 111 or therapy component 113, including but not limited to a determined irradiance parameter and/or a recommended level of irradiance in accordance with a therapy regimen.

Control component 112 may be configured to adjust and/or control the amount of current supplied to set of light sources 11 based on measurements by one or more sensors 142 conveying information related to one or more irradiance levels of electromagnetic radiation emitted by a subset of light sources 11. The subset of light sources that is related and/or used for measurements by one or more sensors 142 may be referred to as the measurement subset (or the measurement subset of light sources). In some embodiments, the measurement subset may include 1 or 2 light sources. In some embodiments, the measurement subset may include 3, 4, 5, or another number of light sources. In some embodiments, the measurement subset includes fewer light sources that set of light sources 11.

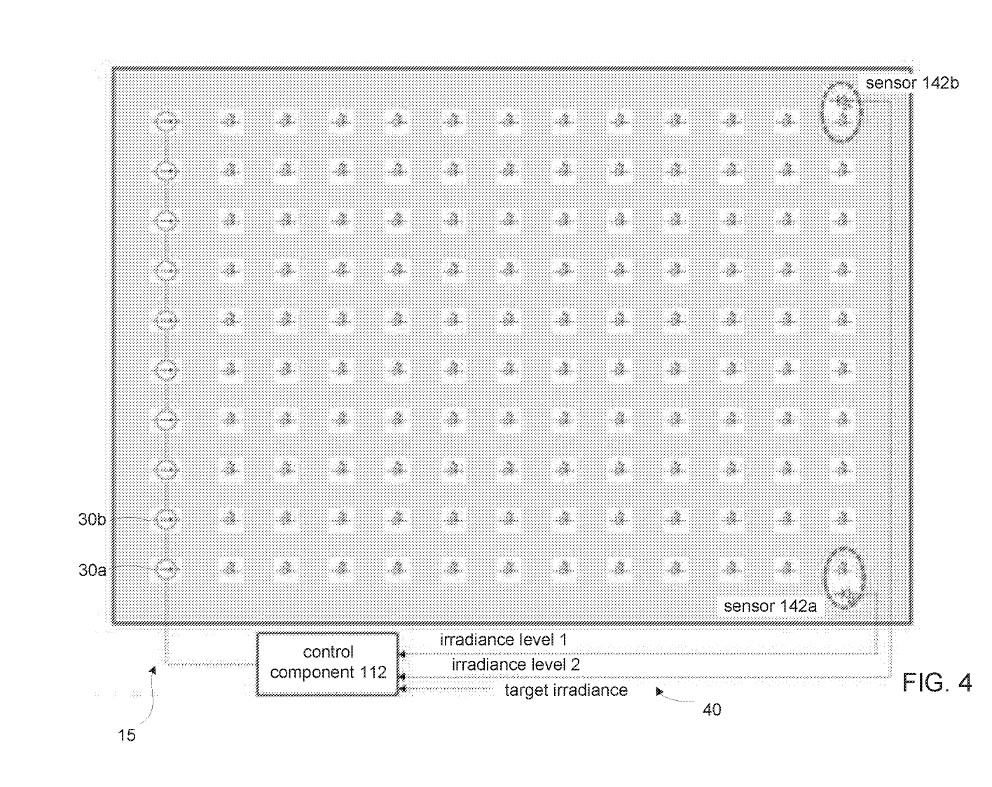

In some embodiments, the one or more light sources 11 are controlled using a circuit 40 of panel 15 depicted in FIG. 4. Circuit 40 includes ten rows of a power supply and twelve light sources 11 each. Two of the power supplies are labeled 30a and 30b. As depicted in FIG. 4, the one or more light sources 11 are light-emitting diodes (LEDs). This is an exemplary configuration. Individual ones of the rows may correspond to rows and/or columns of a grid (and/or triangular pattern) of LEDs as described elsewhere herein. For example, an implementation of a phototherapy panel using more than ten rows and/or columns of more than twelve LEDs each may be implemented by extending circuit 40 as appropriate. Circuit 40 depicts a first sensor 142a and a second sensor 142b (the same as or similar to sensor 142 depicted in FIG. 2). Output signals from sensor 142a convey information related to a first irradiance level (indicated as “irradiance level 1” in FIG. 4). Output signals from sensor 142b convey information related to a second irradiance level (indicated as “irradiance level 2” in FIG. 4). The first and second irradiance level may be used by control component 112 (e.g. in conjunction with a target irradiance corresponding with a therapy regimen) to adjust the amount of current supplied by the power supplies in circuit 40, including but not limited to power supply 30a and power supply 30b.

Operation of circuit 40 may be adjustable to at least two settings, for moderate and high levels of intensity of the phototherapy. For example, a moderate level of intensity may correspond to 15·W/cm²/nm, whereas a high level of intensity may correspond to 30·W/cm²/nm or more. Light sources 11 in circuit 40 are driven by a power supply, which may e.g. supply 24V. Variation of the voltage may vary the current through the rows of circuit 40, and thus through the LEDs therein. Adjusting the voltage may correspond to circuit 40 operating in another setting.

In a preferred embodiment of circuit 40, a voltage of 3V per LED may correspond to a 20 mA current going through the LEDs, which in turn correspond to the high level of intensity of 30·W/cm²/nm for the provided phototherapy. Reducing the voltage corresponds to a different operational setting, with a reduced level of intensity for the provided phototherapy.

In some embodiments, one or more (photo-)sensors (or photodiodes) may be mounted on top of a light source (or LED) in the measurement subset of light sources (e.g. connected by optical glue, which may serve to reduce external factors that might otherwise cause an undue influence for measurements). In some embodiments, an LED and a photodiode may be embedded and/or integrated in a package or housing to improve measurement reliability. In some embodiments, light sources in a corner of a panel or circuit may be suitable as elements in the measurement subset, as light sources in the corner may be less likely to contribute to phototherapy under common usage conditions of system 10 (e.g. based on placing an infant centrally on a panel).

In some embodiments, operation of control component 112 is responsive to, and/or controlled by a timer. For example, phototherapy is stopped after a predetermined period of time has elapsed, as indicated by the timer. In some embodiments, the level of intensity of the provided phototherapy is reduced over time, for example gradually from more to less intensive levels (or settings of control component 112 corresponding thereto). For example, control of the level of intensity may be programmed into an algorithm that operates based on one or more of the current intensity level of emitted electromagnetic radiation 12 as determined through a sensor such as sensor 142a and/or sensor 142b, elapsed time of phototherapy according to a timer, prescribed therapy regimen as provided by a caregiver.

In some embodiments, the measurement subset may include a single LED. However, if this LED, for whatever reason, prematurely indicates a reduced efficiency (which in turn may be counteracted by increasing the amount of current provided to this and other LEDs, and/or which in turn may lead to an indication that the set of light sources 11 needs to be replaced) any subsequent action, determination, or decision may likewise be premature. As an improvement, the measurement could include multiple LEDs. For example, the measurement subset could include two LEDs, such that aberrant measurements may be ignored and related premature actions, determinations, and/or decisions avoided. However, individual LEDs in a measurement subset that includes multiple LEDs may convey conflicting information. In some embodiments, a measurement subset may include three LEDs. Control component 112 may be configured to aggregate measurements from multiple LEDs in the measurement subset. For example, control component 112 may be configured to determine and/or select which two LEDs indicate the most similar irradiance level and ignore the outlier measurements from the third LED. The irradiance levels from the selected LEDs may subsequently be used (e.g. by averaging their respective irradiance levels) as described elsewhere herein to adjust the amount of current supplied to set of light sources 11. Reliability and/or quality of the measurements may be improved by increasing the measurement subset to more than three LEDs and/or by applying additional and/or more advanced techniques to remove outlier measurements from consideration prior to adjustments of the power and/or current supplied as described. For example, the measurement subset could include five LEDs and aim one or more strategies to select which LEDs to ignore and which ones to use to create an aggregate (e.g. averaged) value for a measured irradiance level. One strategy may be to ignore the highest and lowest measured level in a measurement subset, and average the remaining measurements. One strategy may compare the measurements and determine which ones fall outside of the distribution, for example based on standard deviation or other statistical techniques and/or analysis. Other strategies, as well as combined strategies, are considered within the scope of this disclosure.

Referring to FIG. 3, therapy component 113 of system 10 is configured to obtain and/or determine a (recommended) phototherapy regimen for infant 106. A phototherapy regimen is based on one or more of the one or more parameters of electromagnetic radiation, one or more of information related to the size/volume/weight of infant 106, information related to the age of infant 106, information related to previously administered phototherapy to infant 106, information related to medical parameters pertaining to the status of infant 106 (e.g. bilirubin measurements), stated and/or provided information and/or instructions from a user 108 or caregiver, clinician input, guidelines, charts, and/or other information. The recommended phototherapy regimen may in turn be used by control component 112 to control the one or more light sources and/or adjust one or more settings of control component 112. In some embodiments, operation of therapy component 113 is responsive to, and/or controlled by a timer in a manner similar to the described usage of a timer related to control component 112. The irradiance level that corresponds to a recommended therapy regimen may interchangeably be referred to as the target irradiance level and/or target irradiance.

Alert component 114 of system 10 in FIG. 3 is configured to determine whether set of light sources 11 should be replaced. Determinations by alert component 114 may be based on a combination of one or more irradiance parameters (as determined by parameter determination component 111) and one or more amounts of power and/or current supplied to set of light sources 11 during operation of the system. In some embodiments, system 10 includes an indicator configured to indicate whether set of light sources 11 should be replaced. The indication indicated by indicator 25 may be based on the determinations of alert component 114. Indicator 25 may be configured to emit electromagnetic radiation, e.g. by activating an indicator light. Alternatively, and/or simultaneously, indicator 25 may generate a sound to warn users when set of light sources 11 should be replaced. The presence or absence of an indication by indicator 25 may obviate a need or requirement for users to check the irradiance level of system 10 prior to usage. Rather than discarding a set of light sources after a predetermined number of service hours, system 10 may be used to its full potential as long the measured irradiance levels indicate that set of light sources 11 does not (yet) need to be replaced. In some embodiments, determinations by alert component 114 may override operation of other components of system 10, and, for example, force a shut-down of system 10.

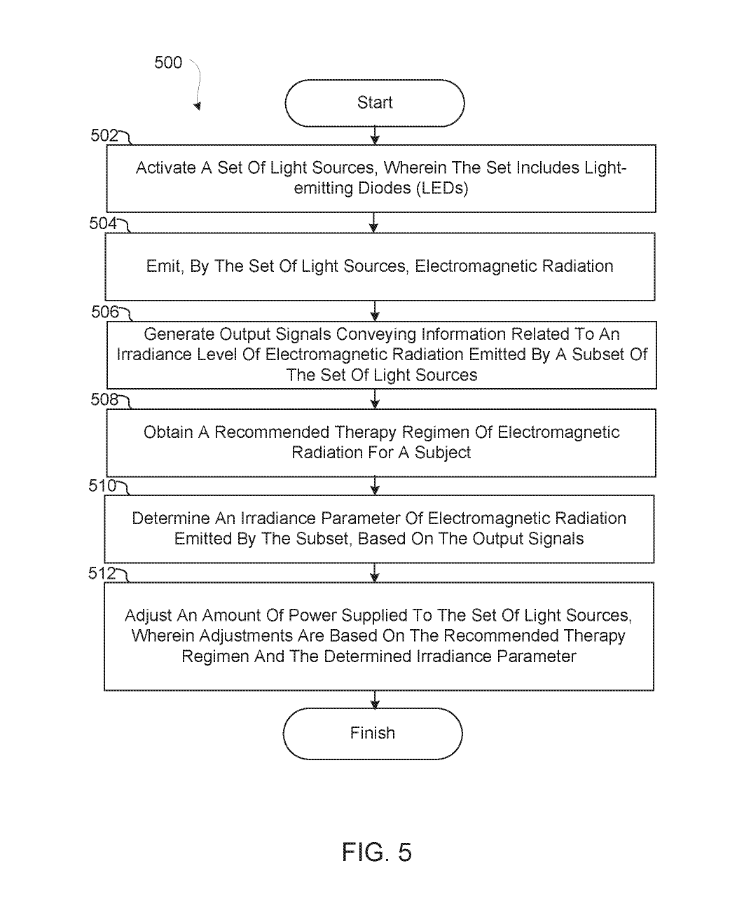

FIG. 5 illustrates a method 500 for providing phototherapy for an infant that is positioned above a transparent or translucent light emitting surface of an infant-supporting body, wherein the infant-supporting body includes a cavity disposed underneath the transparent or translucent light emitting surface. The operations of method 500 presented below are intended to be illustrative. In some embodiments, method 500 is accomplished with one or more additional operations not described, and/or without one or more of the operations discussed. Additionally, the order in which the operations of method 500 are illustrated in FIG. 5 and described below is not intended to be limiting.

In some embodiments, method 500 is implemented in one or more processing devices (e.g., a digital processor, a microcontroller, an analog processor, a digital circuit designed to process information, an analog circuit designed to process information, a state machine, and/or other mechanisms for electronically processing information). The one or more processing devices may include one or more devices executing some or all of the operations of method 500 in response to instructions stored electronically on an electronic storage medium. The one or more processing devices may include one or more devices configured through hardware, firmware, and/or software to be specifically designed for execution of one or more of the operations of method 500.

At an operation 502, a set of light sources is activated. The set includes light-emitting diodes (LEDs. In some embodiments, operation 502 is performed by a set of light source the same as or similar to set of light sources 11 (shown in FIG. 3 and described herein).

At an operation 504, electromagnetic radiation is emitted. In some embodiments, operation 504 is performed by a set of light source the same as or similar to set of light sources 11 (shown in FIG. 3 and described herein).

At an operation 506, output signals are generated that convey information related to an irradiance level of electromagnetic radiation emitted by a subset of light sources from the set of light sources. In some embodiments, operation 506 is performed by a photo-sensor the same as or similar to photo-sensor 142 (shown in FIG. 3 and described herein).

At an operation 508, a recommended therapy regimen of electromagnetic radiation is obtained for a subject. In some embodiments, operation 508 is performed by a therapy component the same as or similar to therapy component 113 (shown in FIG. 3 and described herein).

At an operation 510, an irradiance parameter of electromagnetic radiation emitted by the subset of light sources is determined. The irradiance parameter is based on the output signals. In some embodiments, operation 510 is performed by a parameter determination component the same as or similar to parameter determination component 111 (shown in FIG. 2 and described herein).

At an operation 512, an amount of power supplied to the set of light sources is adjusted. Adjustments are based on the recommended therapy regimen and the determined irradiance parameter. In some embodiments, operation 512 is performed by a control component the same as or similar to control component 112 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination. [55] Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to

1. A system comprising:
a set of light sources configured to emit electromagnetic radiation, wherein the set includes light-emitting diodes (LEDs);
a photo-sensor configured to generate output signals conveying information related to an irradiance level of electromagnetic radiation emitted by a subset of light sources from the set of light sources; and
one or more processors configured to:
obtain a recommended therapy regimen of electromagnetic radiation for a subject,
determine an irradiance parameter of electromagnetic radiation emitted by the subset of light sources, wherein the irradiance parameter is based on the generated output signals, and
adjust an amount of power supplied to the set of light sources, wherein adjustments are based on the recommended therapy regimen and the determined irradiance parameter.

2. The system of claim 1, the subject being an infant, wherein the set of light sources is arranged in a phototherapy panel configured to provide phototherapy to the infant to treat jaundice, responsive to the system being arranged in suitable proximity to the infant.

3. The system of claim 1, further comprising:
a power supply that supplies an amount of current to the set of light sources upon activation, wherein the one or more processors are configured to adjust the amount of power by adjusting the amount of current supplied to the set of light sources to maintain the irradiance parameter at a level that corresponds to the recommended therapy regimen.

4. The system of claim 1, wherein the subset of light sources includes less than three light-emitting diodes (LEDs), wherein the set of light sources includes more than three light-emitting diodes, wherein the photo-sensor includes a photo-diode that is mounted on top of an individual one of the less than three light-emitting diodes (LEDs), and wherein the one or more processors are further configured such that a decrease in the determined irradiance parameter is counteracted by an increase in the amount of power supplied to the set of light sources.

5. The system of claim 3, further comprising:
an indicator configured to indicate a notification, wherein the notification includes emission of electromagnetic radiation,
wherein the one or more processors are further configured to determine whether the set of light sources should be replaced, wherein the determination is based on a combination of the irradiance parameter and the amount of current supplied to the set of light sources during operation of the system,
wherein the notification reflects the determination whether the set of light sources should be replaced.

6. A method of adjusting an amount of power supplied to a set of light sources, the method comprising:
activating a set of light sources, wherein the set includes light-emitting diodes (LEDs);
emitting, by the set of light sources, electromagnetic radiation responsive to activation;
generating output signals conveying information related to an irradiance level of electromagnetic radiation emitted by a subset of light sources from the set of light sources;
obtaining a recommended therapy regimen of electromagnetic radiation for a subject;
determining an irradiance parameter of electromagnetic radiation emitted by the subset of light sources, wherein the irradiance parameter is based on the output signals; and
adjusting an amount of power supplied to the set of light sources, wherein adjustments are based on the recommended therapy regimen and the determined irradiance parameter.

7. The method of claim 6, the subject being an infant, the method further comprising:
arranging the set of light sources in a phototherapy panel,
wherein emitting the electromagnetic radiation provides phototherapy to the infant to treat jaundice, responsive to the set of light sources being arranged in suitable proximity to the infant.

8. The method of claim 6, further comprising;
supplying an amount of current to the set of light sources upon activation,
wherein adjusting the amount of power includes adjusting the amount of current supplied to the set of light sources to maintain the irradiance parameter at a level that corresponds to the recommended therapy level.

9. The method of claim 6, wherein the subset of light sources includes less than three light-emitting diodes (LEDs), wherein the set of light sources includes more than three light-emitting diodes, wherein the photo-sensor includes a photo-diode that is mounted on top of an individual one of the less than three light-emitting diodes (LEDs), and wherein adjusting the amount of power includes counteracting a decrease in the determined irradiance parameter by an increase in the amount of power supplied to the set of light sources.

10. The method of claim 8, further comprising:
determining whether the set of light sources should be replaced based on a combination of the irradiance parameter and the amount of current supplied to the set of light sources during operation of the system; and
indicating a notification that reflects the determination whether the set of light sources should be replaced, wherein the notification includes emission of electromagnetic radiation.

11. A system configured to adjust an amount of power supplied to a set of light sources, the system comprising:
emitting means for emitting electromagnetic radiation, wherein the emitting means includes light-emitting diodes (LEDs);
means for generating output signals conveying information related to an irradiance level of electromagnetic radiation emitted by a portion of the emitting means;
means for obtaining a recommended therapy regimen of electromagnetic radiation for a subject;
means for determining an irradiance parameter of electromagnetic radiation emitted by the portion of the emitting means, wherein the irradiance parameter is based on the output signals; and
means for adjusting an amount of power supplied to the means for emitting, wherein adjustments are based on the recommended therapy regimen and the determined irradiance parameter.

12. The system of claim 11, the subject being an infant, the system further comprising:
means for carrying the emitting means and providing phototherapy to the infant to treat jaundice, responsive the system being arranged in suitable proximity to the infant.

13. The system of claim 11, further comprising:
means to supply an amount of current to the emitting means upon activation,
wherein the means for adjusting the amount of power is configured to adjust the amount of power by adjusting the amount of current supplied to the emitting means to maintain the irradiance parameter at a level that corresponds to the recommended therapy regimen. includes light-emitting diodes (LEDs)

14. The system of claim 11, wherein the portion of the emitting means includes less than three light-emitting diodes (LEDs), wherein the emitting means includes more than three light-emitting diodes, wherein the means for generating output signals includes a photo-diode that is mounted on top of an individual one of the less than three light-emitting diodes (LEDs), and wherein the means for adjusting the amount of power is configured such that a decrease in the determined irradiance parameter is counteracted by an increase in the amount of power supplied to the emitting means.

15. The system of claim 13, further comprising;
means for determining whether the emitting means should be replaced based on a combination of the irradiance parameter and the amount of current supplied to the emitting means during operation of the system; and
means for indicating a notification that reflects the determination whether the emitting means should be replaced, wherein the notification includes emission of electromagnetic radiation.
